SEQUENCE LISTING (62 sequences)

SEQ ID NO: 1 - protein, 7 aa, Artificial Sequence
  inserted amino acid section
THGTPAD

SEQ ID NO: 2 - protein, 7 aa, Artificial Sequence
  inserted amino acid section
GCGGIPE

SEQ ID NO: 3 - protein, 7 aa, Artificial Sequence
  inserted amino acid section
QHEALRC

SEQ ID NO: 4 - protein, 7 aa, Artificial Sequence
  inserted amino acid section
DVPTTGI

SEQ ID NO: 5 - protein, 7 aa, Artificial Sequence
  inserted amino acid section
DTRCPSS

SEQ ID NO: 6 - protein, 7 aa, Artificial Sequence
  inserted amino acid section
ALLCRHD

SEQ ID NO: 7 - protein, 7 aa, Artificial Sequence
  inserted amino acid section
GSLQSGE

SEQ ID NO: 8 - protein, 7 aa, Artificial Sequence
  inserted amino acid section
DPPSSSA

SEQ ID NO: 9 - protein, 7 aa, Artificial Sequence
  inserted amino acid section
VSSTSPR

SEQ ID NO: 10 - protein, 7 aa, Artificial Sequence
  inserted amino acid section
VSSTPPR

SEQ ID NO: 11 - protein, 7 aa, Artificial Sequence
  inserted amino acid section
NLPGSGD

SEQ ID NO: 12 - protein, 7 aa, Artificial Sequence
  inserted amino acid section
APSESPN

SEQ ID NO: 13 - protein, 7 aa, Artificial Sequence
  inserted amino acid section
GIEIGCS

SEQ ID NO: 14 - protein, 7 aa, Artificial Sequence
  inserted amino acid section
VGPSRGS

SEQ ID NO: 15 - protein, 7 aa, Artificial Sequence
  inserted amino acid section
SNGNACG

SEQ ID NO: 16 - protein, 7 aa, Artificial Sequence
  inserted amino acid section
AERQPTG

SEQ ID NO: 17 - protein, 7 aa, Artificial Sequence
  inserted amino acid section
GGELDCR

SEQ ID NO: 18 - protein, 7 aa, Artificial Sequence
  inserted amino acid section
ANYSPPA

SEQ ID NO: 19 - protein, 7 aa, Artificial Sequence
  inserted amino acid section
TGSPCTA

SEQ ID NO: 20 - protein, 7 aa, Artificial Sequence
  inserted amino acid section
MRTGVSV

SEQ ID NO: 21 - protein, 7 aa, Artificial Sequence
  inserted amino acid section
SDWTEDP

SEQ ID NO: 22 - protein, 7 aa, Artificial Sequence
  inserted amino acid section
GSPGDAG

SEQ ID NO: 23 - protein, 7 aa, Artificial Sequence
  inserted amino acid section
QSAGLEC

SEQ ID NO: 24 - protein, 7 aa, Artificial Sequence
  inserted amino acid section
SQNSTSR

SEQ ID NO: 25 - protein, 7 aa, Artificial Sequence
  inserted amino acid section
CPYQPPG

SEQ ID NO: 26 - protein, 7 aa, Artificial Sequence
  inserted amino acid section
ARDSGHT

SEQ ID NO: 27 - protein, 7 aa, Artificial Sequence
  inserted amino acid section
YPPPCES

SEQ ID NO: 28 - protein, 7 aa, Artificial Sequence
  inserted amino acid section
SPGQSCW

SEQ ID NO: 29 - protein, 7 aa, Artificial Sequence
  inserted amino acid section
GNGAGAH

SEQ ID NO: 30 - protein, 7 aa, Artificial Sequence
  inserted amino acid section
GCAGGNY

SEQ ID NO: 31 - protein, 7 aa, Artificial Sequence
  inserted amino acid section
VGSTLPQ

SEQ ID NO: 32 - protein, 7 aa, Artificial Sequence
  inserted amino acid section
YGARHDG

SEQ ID NO: 33 - protein, 7 aa, Artificial Sequence
  inserted amino acid section
DCTPGAS

SEQ ID NO: 34 - protein, 7 aa, Artificial Sequence
  inserted amino acid section
CFPRPDE

SEQ ID NO: 35 - protein, 7 aa, Artificial Sequence
  inserted amino acid section
FDPGYRS

SEQ ID NO: 36 - protein, 7 aa, Artificial Sequence
  inserted amino acid section
ANHGVTR

SEQ ID NO: 37 - protein, 7 aa, Artificial Sequence
  inserted amino acid section
DSVSLGA

SEQ ID NO: 38 - protein, 7 aa, Artificial Sequence
  inserted amino acid section
AGNQTRS

SEQ ID NO: 39 - protein, 7 aa, Artificial Sequence
  inserted amino acid section
GVPQRPE

SEQ ID NO: 40 - protein, 7 aa, Artificial Sequence
  inserted amino acid section
EPSGSVC

SEQ ID NO: 41 - protein, 7 aa, Artificial Sequence
  inserted amino acid section
ELHSPSA

SEQ ID NO: 42 - protein, 7 aa, Artificial Sequence
  inserted amino acid section
PNEGAGR

SEQ ID NO: 43 - protein, 7 aa, Artificial Sequence
  inserted amino acid section
SGSPTHC

SEQ ID NO: 44 - protein, 7 aa, Artificial Sequence
  inserted amino acid section
MPRCTEA

SEQ ID NO: 45 - protein, 7 aa, Artificial Sequence
  inserted amino acid section
TGSPYTA

SEQ ID NO: 46 - protein, 7 aa, Artificial Sequence
  inserted amino acid section
NLTRPAL

SEQ ID NO: 47 - protein, 7 aa, Artificial Sequence
  inserted amino acid section
SPTRDPC

SEQ ID NO: 48 - protein, 7 aa, Artificial Sequence
  inserted amino acid section
YAPARSS

SEQ ID NO: 49 - protein, 7 aa, Artificial Sequence
  inserted amino acid section
VPVRPTS

SEQ ID NO: 50 - protein, 7 aa, Artificial Sequence
  inserted amino acid section
RVGHGSA

SEQ ID NO: 51 - protein, 7 aa, Artificial Sequence
  inserted amino acid section
RISTEGA

SEQ ID NO: 52 - protein, 7 aa, Artificial Sequence
  inserted amino acid section
QQTGGTR

SEQ ID NO: 53 - protein, 7 aa, Artificial Sequence
  inserted amino acid section
DTMPSGV

SEQ ID NO: 54 - protein, 747 aa, Artificial Sequence
  CHAIN 1..587: N-terminal section of native CAP
  CAP protein with inserted amino acid section
  CHAIN 588..590: linker
  CHAIN 591..597: inserted amino acid sequence
  CHAIN 598..599: linker
  CHAIN 600..747: C-terminal section of CAP
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNASATHGTPADAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 55 - protein, 747 aa, Artificial Sequence
  CHAIN 1..587: N-terminal section of native CAP
  CAP with inserted amino acid section
  CHAIN 588..590: linker
  CHAIN 591..597: inserted amino acid sequence
  CHAIN 598..599: linker
  CHAIN 600..747: C-terminal section of CAP
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNASANLPGSGDAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 56 - protein, 735 aa, Adeno-associated virus - 2 [sequence omitted]

SEQ ID NO: 57 - DNA, 18 bp, Artificial Sequence
  forward primer in CAP gene
ggtacgacgacgattgcc

SEQ ID NO: 58 - DNA, 18 bp, Artificial Sequence
  reverse primer CAP gene
atgtccgtccgtgtgtgg

SEQ ID NO: 59 - DNA, 22 bp, Artificial Sequence
  Ptbp2 forward primer
tctccattccctatgttcatgc

SEQ ID NO: 60 - DNA, 22 bp, Artificial Sequence
  Ptbp2 reverse primer
gttcccgcagaatggtgaggtg

SEQ ID NO: 61 - DNA, 20 bp, Artificial Sequence
  Ptbp2 binding probe
atgttcctcggaccaacttg

SEQ ID NO: 62 - protein, 7 aa, Artificial Sequence
  comparative inserted amino acid section
LPSRPSL

(19) United States
(12) Patent Application Publication
(10) Pub. No.: US 2023/0074198 A1
(43) Pub. Date: Mar. 9, 2023

(54) Viral vector particle based on AA V2 for gene therapy

(71) Applicant: Medizinische Hochschule Hannover, Hannover (DE)

(72) Inventors: Thomas Thum, Hannover (DE); Hildegard Büning, Hannover (DE); Laura Rode, Hannover (DE); Christian Bär, Hannover (DE)

(21) Appl. No.: 17/800,338

(22) PCT Filed: Feb. 22, 2021
(86) PCT No.: PCT/EP2021/054347

§ 371 Date: Aug. 17, 2022

(30) Foreign Application Priority Data
Feb. 20, 2020 (EP) 20158633.6

(51) Int. Cl. C12N 15/86

(52) U.S. Cl. CPC C12N 15/86; C12N 2750/14143; C12N 2750/14122; A61K 48/00

(57) ABSTRACT

The invention provides a viral vector particle based on AAV2, which in its capsid protein (CAP) contains an inserted amino acid section which confers tropism for cardiomyocytes.

The present invention relates to a viral vector particle which is based on adeno-associated virus serotype 2 (AAV2) for use in gene therapy, especially for use in the treatment of cardiac diseases including cardiac defects, e.g. for use in the genetic treatment of cardiac diseases including cardiac defects. The cardiac disease or defect preferably is a disease or defect of cardiomyocytes. The cardiac disease or defect is a medical condition which is e.g. associated with cardiac overload, or cardiac insufficiency, e.g. the viral vector particle is for use in the treatment of the following medical indications: delivery of a transgene which exhibits therapeutic effects in cardiomyocytes after myocardial infarctions, cardiac hypertrophy or for the treatment of heart failure.

The viral vector particle of the invention has the advantage of being more specific for cardiomyocytes and being less efficient in transducing non-target cells, e.g. liver cells, when compared to wild-type AAV serotype 2 and 9 (AAV2 and AAV9), and to allow for expression of a transgene encoded in the viral vector particle in cardiomyocytes.

### STATE OF THE ART

Perabo et al., Molecular Therapy, Vol. 8, No. 1, 151-157 (2003) describe the insertion of a random sequence of 7 amino acids into capsid proteins at position 587 referred to the VP1 capsid protein of the AAV2 virion and selection of AAV2 mutants from the human megakaryocytic cell line M-07e or B-cell chronic lymphocytic leukemia cell line Mecl that was co-infected with adenovirus. As a result, a sequence of the mutant capsid protein was identified that conferred receptor specificity, but not cell specificity, to the viral vector.

Ying et al., Gene Therapy 17, 980-990 (2010) describe three rounds of screening of an AAV2 display peptide library for selecting vectors having higher specificity for heart tissue by injecting the AAV2 library into a mouse, isolating heart tissue slices from the mouse 3 days afterwards, and in vitro super-infecting the heart tissue slices with Ad5 in cultivation conditions. Two AAV2 variants were identified that showed increased specificity for heart tissue, but reporter gene expression from these variants was lower than expression from a wild-type AAV9 viral vector particle.

Wang et al., Nature Reviews Drug Discovery 358-378 (2019) quote that recombinant AAV (AAV vector particles or AAV vectors) in their single stranded DNA can consist of non-viral sequences flanked by the viral ITRs, so that the only viral sequences are the ITRs. The ITRs of AAV serve for genome replication and as a packaging signal during vector production.

Rockman et al., PNAS 88: 8277-8281 (1991) describe the induction of cardiac pressure overload in mice for generating a murine model for cardiac hypertrophy.

Zhang et al., Hum. Gene Ther., 1284-1296, doi: 10.1089/hu.2019/027 (2019), describe a plasmid termed pRC'99 which contains the open reading frames (ORFs) for the rep and cap proteins of AAV2 for use in cloning and generating AAV vector particles of capsid variants.

Zincarelli et al., Molecular Therapy, Vol. 16, No. 6, 1073-1080 (2008) have shown that within the naturally occurring serotypes of AAVs, AAV9 shows the highest transgene expression in heart in mice after systemic injection.

As reviewed by Hajjar & Ishikawa, Circulation Res, Vol. 120, No. 1, 33-35 (2017), the AAV9 emerged as the vector with high cardiac tropism and it thus typically used to target the heart in gene therapy research.

### OBJECT OF THE INVENTION

It is an object of the invention to provide alternative viral vector particles based on AAV2, especially capsid proteins of AAV2, which have improved specificity, also called tropism, for mouse and for human cardiomyocytes and lower affinity for liver tissue, e.g. compared to wild-type serotypes AAV2, and especially compared to AAV9. For use in gene therapy, the viral vector particle with good specificity for cardiomyocytes should allow expression of a nucleic acid coding sequence that is contained in the viral vector particle in cardiomyocytes.

### DESCRIPTION OF THE INVENTION

The invention achieves the object by the features of the claims, and especially provides a viral vector particle based on AAV2, which in its capsid protein (CAP) C-terminally to amino acid No. 587, and/or C-terminally to amino acid No. 588, and/or C-terminally to amino acid No. 453 of the wild-type amino acid sequence of CAP contains an inserted amino acid section comprising or consisting of one of the following amino acid sequences selected from SEQ ID NO: 1 to SEQ ID NO: 53, preferably one of SEQ ID NO: 1 or SEQ ID NO: 11. The CAP of the invention, from N-terminus to C-terminus comprises or consists of the N-terminal section of CAP, which comprises or consists of amino acids 1 to 587 of the wild-type CAP, optionally a linker sequence, the inserted amino acid section, optionally a linker sequence, and the C-terminal section of CAP, which for the insertion C-terminally to amino acid No. 587 comprises or consists of amino acids 588 to 735 of the wild-type CAP, for the insertion C-terminally to amino acid No. 588 comprises or consists of amino acids 589 to 735 of the wild-type CAP, or for the insertion C-terminally to amino acid No. 453 comprises or consists of amino acids 454 to 735 of the wild-type CAP. Accordingly, the inserted amino acid section in one embodiment is inserted between amino acids N587 and R588 of the wild-type CAP amino acid sequence. In the alternative, the inserted amino acid sequence is inserted between amino acids R588 and 589 of the wild-type CAP amino acid sequence, which alternative herein is also described by the insertion between N587 and R588 and/or described by the insertion between 1456 and amino acid 454 of the wild-type CAP amino acid sequence, and all features from the description and claims apply to both the insertion between N587 and R588 of the wild-type CAP amino acid sequence, and apply to the insertion between R588 and 589, and apply to the insertion between amino acids 1453 and 454, each amino acid numbering in respect of the wild-type CAP amino acid sequence. For each of these insertion sites, especially in the embodiment in which the inserted amino acid section is inserted between amino acids No. 453 and 454 of the wild-type CAP amino acid sequence, it is preferred that the CAP amino acid sequence is additionally mutated to R585A (amino acid 585 Arg to Ala) and R588A (amino acid 588 Arg to Ala). These preferred additional mutations disturb the heparin sulfate proteoglycan binding site of wild-type CAP.

SEQ ID NO: 1
THGTPAD
SEQ ID NO: 2
GCGGIPE
SEQ ID NO: 3
QHEALRC
SEQ ID NO: 4
DVPTTGI
SEQ ID NO: 5
DTRCPSS
SEQ ID NO: 6
ALLCRHD
SEQ ID NO: 7
GSLQSGE
SEQ ID NO: 8
DPPSSSA
SEQ ID NO: 9
VSSTSPR
SEQ ID NO: 10
VSSTPPR
SEQ ID NO: 11
NLPGSGD
SEQ ID NO: 12
APSESPN
SEQ ID NO: 13
GIEIGCS
SEQ ID NO: 14
VGPSRGS
SEQ ID NO: 15
SNGNACG
SEQ ID NO: 16
AERQPTG
SEQ ID NO: 17
GGELDCR
SEQ ID NO: 18
ANYSPPA
SEQ ID NO: 19
TGSPCTA
SEQ ID NO: 20
MRTGVSV
SEQ ID NO: 21
SDWTEDP
SEQ ID NO: 22
GSPGDAG
SEQ ID NO: 23
QSAGLEC
SEQ ID NO: 24
SQNSTSR
SEQ ID NO: 25
CPYQPPG
SEQ ID NO: 26
ARDSGHT
SEQ ID NO: 27
YPPPCES
SEQ ID NO: 28
SPGQSCW
SEQ ID NO: 29
GNGAGAH
SEQ ID NO: 30
GCAGGNY
SEQ ID NO: 31
VGSTLPQ
SEQ ID NO: 32
YGARHDG
SEQ ID NO: 33
DCTPGAS
SEQ ID NO: 34
CFPRPDE
SEQ ID NO: 35
FDPGYRS
SEQ ID NO: 36
ANHGVTR
SEQ ID NO: 37
DSVSLGA
SEQ ID NO: 38
AGNQTRS
SEQ ID NO: 39
GVPQRPE
SEQ ID NO: 40
EPSGSVC
SEQ ID NO: 41
ELHSPSA
SEQ ID NO: 42
PNEGAGR
SEQ ID NO: 43
SGSPTHC
SEQ ID NO: 44
MPRCTEA
SEQ ID NO: 45
TGSPYTA
SEQ ID NO: 46
NLTRPAL
SEQ ID NO: 47
SPTRDPC
SEQ ID NO: 48
YAPARSS
SEQ ID NO: 49
VPVRPTS
SEQ ID NO: 50
RVGHGSA
SEQ ID NO: 51
RISTEGA
SEQ ID NO: 52
QQTGGTR
SEQ ID NO: 53
DTMPSGV

Therein, between amino acid No. 587 of CAP, or amino acid 588 of CAP, or amino acid 453 of CAP, respectively, and the N-terminal amino acid of the inserted amino acid section no additional amino acid may be present so that the inserted amino acid sequence is directly adjacent to amino acid No. 587 of CAP, respectively adjacent to amino acid No. 588 of CAP, or respectively adjacent to amino acid No. 454 of CAP, or alternatively a linker sequence of 1 to 5 amino acids, e.g. of 1 to 4 amino acids may be arranged between amino acid No. 587 of CAP, respectively amino acid No. 588 of CAP, or respectively amino acid No. 454 of CAP, and the inserted amino acid section, and/or wherein between the C-terminus of the inserted amino acid section and the remaining C-terminal portion of CAP, no additional amino acid may be present so that the inserted amino acid sequence is directly adjacent to the amino acid of the C-terminal portion of CAP, and/or a linker sequence of 1 to 4 amino acids, e.g. of 1 to 3 amino acids may be arranged between the C-terminus of the inserted amino acid section and the N-terminal amino acid of the remaining C-terminal portion of CAP.

For the insertion C-terminally to amino acid No. 587, the N-terminal amino acid of the remaining C-terminal portion of CAP preferably is amino acid No. 588 of the wild-type CAP. Preferably, the C-terminal portion of CAP, which is adjacent to the C-terminus of the inserted amino acid section, optionally with a linker sequence between the C-terminus of the inserted amino acid section and the C-terminal portion of CAP, has the amino acid sequence of amino acids No. 588 to No. 735 of SEQ ID NO: 56.

For the insertion C-terminally to amino acid No. 588, the N-terminal amino acid of the remaining C-terminal portion of CAP preferably is amino acid No. 589 of the wild-type CAP. Preferably, the C-terminal portion of CAP, which is adjacent to the C-terminus of the inserted amino acid section, optionally with a linker sequence between the C-terminus of the inserted amino acid section and the C-terminal portion of CAP, has the amino acid sequence of amino acids No. 589 to No. 735 of SEQ ID NO: 56.

For the insertion C-terminally to amino acid No. 454, the N-terminal amino acid of the remaining C-terminal portion of CAP preferably is amino acid No. 455 of the wild-type CAP. Preferably, the C-terminal portion of CAP, which is adjacent to the C-terminus of the inserted amino acid section, optionally with a linker sequence between the C-terminus of the inserted amino acid section and the C-terminal portion of CAP, has the amino acid sequence of amino acids No. 455 to No. 735 of SEQ ID NO: 56.

The inserted amino acid section can be directly adjacent to the C-terminus of amino acid No. 587, respectively directly adjacent to the C-terminus of amino acid No. 588, or respectively directly adjacent to the C-terminus of amino acid No. 455 of the wild-type amino acid sequence of CAP, or a linker sequence, e.g. of 1 to 5 amino acids, preferably of 3 amino acids, can be arranged between amino acid No. 587, respectively amino acid No. 588, or respectively amino acid No. 453 of the wild-type amino acid sequence of CAP and the inserted amino acid section. The inserted amino acid can be directly adjacent to the N-terminus of amino acid No. 588, respectively amino acid No. 589, or respectively amino acid No. 454 of the wild-type amino acid sequence of CAP, or a linker sequence, e.g. of 1 to 4 amino acids, preferably of 2 amino acids, can be arranged between the inserted amino acid section and the N-terminus of amino acid No. 588, respectively amino acid No. 589, or respectively amino acid No. 454 of the wild-type amino acid sequence of CAP. An exemplary linker sequence for arrangement between amino acid No. 588, respectively amino acid No. 589, or respectively amino acid No. 454 of the wild-type amino acid sequence of CAP and the N-terminus of the inserted amino acid section is ASA, an exemplary linker sequence for arrangement between amino acid No. 588, respectively amino acid No. 589, or respectively amino acid No. 454 of the wild-type amino acid sequence of CAP and the C-terminus of the inserted amino acid section is AA. Preferably, the inserted amino acid section together with a linker sequence at its N-terminus and a linker sequence at its C-terminus consists of 16 to 7 amino acids, e.g. 14 to 7 amino acids, more preferably of 12 amino acids.

The linker section in each case can comprise or consist of at least one of the amino acids selected from Ala, Thr, Pro, Gly, Leu, and/or Ser, which amino acids can be different from one another or all the same in each linker section.

The viral vector particles of the invention have the advantage of increased tropism, or specificity, for cardiomyocytes, e.g. in comparison to AAV2 and AAV9 having respective wild-type CAP, of reduced tropism for other cell-types than cardiomyocytes, especially reduced tropism for liver tissue, and of expression of the nucleic acid sequence that is contained in the viral vector particle in cardiomyocytes, e.g. transgene expression at a level comparable to the level of expression of the same transgene from AAV9 and much higher than the level of expression of the same transgene from AAV2. Cardiomyocytes are part of cardiac tissue, and the viral vector particle can be for medical use, e.g. for use in the treatment of cardiac diseases or cardiac defects, e.g. for administration to a human patient. Further, the viral vector particles have the advantage of allowing its production in cells at a high titer.

Further, it was found that the viral vector particles according to the invention were neutralized to a significantly lower extent by human intravenous immunoglobulin (IVIG), which antibodies are present in the majority of persons, and which IVIG is known to neutralize wild-type AAV particles. This shows that the viral vector particles according to the invention for use in treatment have the advantage of evading neutralization by naturally occurring antibodies against AAV.

Viral vector particles containing a CAP including one of the inserted amino acid sections of SEQ ID NO: 1 to SEQ ID NO: 53, inserted between amino acid No. 587 and amino acid No. 588 of the wild-type CAP, and/or inserted between amino acid No. 588 and amino acid No. 589 of the wild-type CAP, and/or inserted between amino acid No. 453 and amino acid No. 454 of the wild-type CAP, preferably one of SEQ ID NO: 1 or SEQ ID NO: 11, have a high tropism for cardiomyocytes, allow for expression of nucleic acid sequences contained in the particles in cardiomyocytes, and significantly less expression in liver tissue, e.g. than AAV9 viral vector particles containing the same nucleic acid sequences encoding a transgene.

Accordingly, it is currently assumed that the viral particles having a CAP containing one of the inserted amino acid sections have affinity to the same cardiomyocyte surface molecule which acts as a target molecule.

The amino acid section inserted between amino acid No. 587 and amino acid No. 588 of the wild-type CAP, i.e. to the C-terminus of amino acid N. 587 of the wild-type CAP, for the preferred inserted amino acid section of SEQ ID NO: 1 is contained as an insert in the wild-type CAP in SEQ ID NO: 54, and the preferred inserted amino acid section of SEQ ID NO: 11 is contained as an insert in the wild-type CAP in SEQ ID NO: 55, wherein for each inserted amino acid section a linker sequence of amino acids ASA is arranged between the N-terminal section of the wild-type CAP, i.e. between amino acid No. 587, and a linker sequence of amino acids AA is arranged between the inserted amino acid section and the C-terminal section of CAP, i.e. between the inserted amino acid section and amino acid 588 of the wild-type CAP. The wild-type CAP is encoded by the AAV2 cap ORF (coding sequence is SEQ ID NO: 56). In the alternative to ASA, the linker arranged between the N-terminal section of the wild-type CAP, i.e. between amino acid No. 587 of wild-type CAP, and the inserted amino acid section, the linker can have the amino acid sequence AAA.

The viral vector particles contain a nucleic acid construct, e.g. a sense strand or an antisense strand of single-stranded DNA, comprising or consisting of an effector sequence between terminal ITR sequences of AAV2. The effector sequence can be an expression cassette encoding an effector molecule, e.g. encoding a functional non-coding RNA or a protein-coding RNA, which can be expressed from the vector construct in cardiomyocytes and thereby exhibits a therapeutic beneficial function in cardiomyocytes and thus also for the whole heart.

Herein, transduction of cardiomyocytes is the introduction of nucleic acids by the viral vector particles into cardiomyocytes, which process can also be referred to as infection by the viral vector particles, especially for use of the viral vector particles in the treatment of cardiomyocytes. Generally, the viral vector particles can be for use in the treatment of cardiomyocytes, in vivo or in vitro, e.g. for the treatment of genetic defects of cardiomyocytes, especially for transduction of cardiomyocytes.

An exemplary effector molecule can be selected from any wild-type sequence, e.g. for use in complementing a defective gene in the recipient of the viral vector particle. Exemplary effector molecules are natural genes, including genes from any species, preferably human genes.

In a specific embodiment, empty viral particles are provided, which do not contain a nucleic acid molecule. These empty viral particles can e.g. be associated with a functional molecule for delivery of the functional molecule to heart tissue. The functional molecule can e.g. be a therapeutic agent or an indicator compound, e.g. a dye or a pharmaceutically acceptable diagnostic contrast agent, or a combination of at least two of these. Empty viral particles can be produced in HEK293 cells. The process comprised the steps of transfecting the respective helper plasmid (containing the cap gene, if applicable with an inserted amino acid section, and the rep gene of wild-type AAV2) and an adenoviral helper plasmid (containing adenoviral helper functions required for AAV vector production) in HEK293 cells for AAV empty capsid production (no vector genome plasmid containing ITRs flanking the expression cassette is used in this specific case), with subsequent purification of empty capsid particles by iodixanol gradient centrifugation.

Generally, the process for producing AAV viral vector particles according to the invention can be by delivery, e.g. by plasmid transfection with or without helper virus co-infection, of all required components for AAV vector production, e.g. from a vector genome containing the transgene expression cassette flanked by ITRs, AAV rep and AAV cap genes as well as other viral helper genes necessary for AAV particle production, e.g. from adenovirus. The process can be performed in a cultivated eukaryotic host cell, followed by cell lysis and removal of cellular components and plasmid DNA, e.g. by enzymatic digestion, filtration and/or centrifugation, and further purification, e.g. by gradient density centrifugation and/or chromatography of AAV viral vector particles. The AAV viral vector particles obtained by the process comprise the capsid protein (CAP) which C-terminally to amino acid No. 587 of the wild-type amino acid sequence of CAP contains an inserted amino acid section comprising one of the amino acid sequences selected from SEQ ID NO: 1 to SEQ ID NO: 53.

The viral vector particles can be formulated for injection, e.g. for direct injection into heart tissue, or for systemic injection, e.g. intravenous (i.v.) injection.

The invention is now described by way of an example and with reference to the figures, which show in

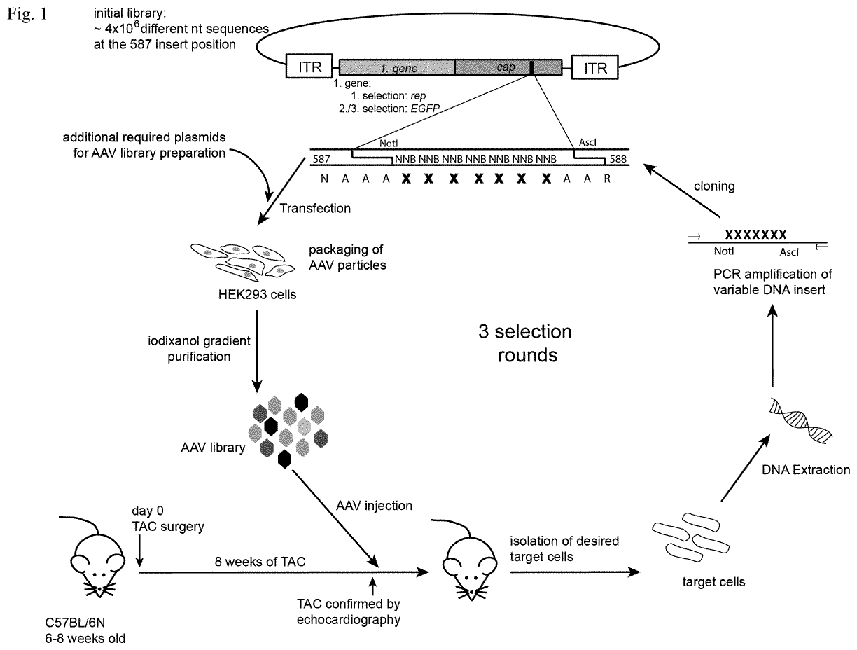

FIG. 1 a scheme of the selection process used for identifying CAPs with inserted amino acid sections of the invention,

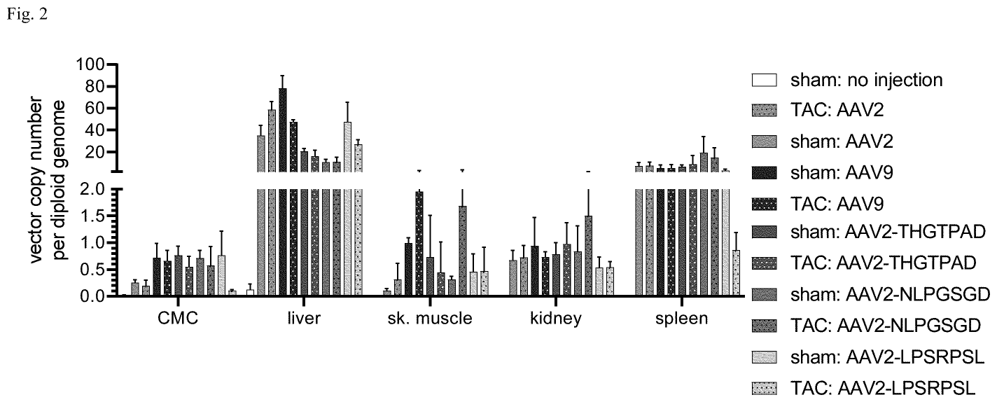

FIG. 2 shows copy numbers of viral vector particles present in different cell types of experimental animals after systemic injection,

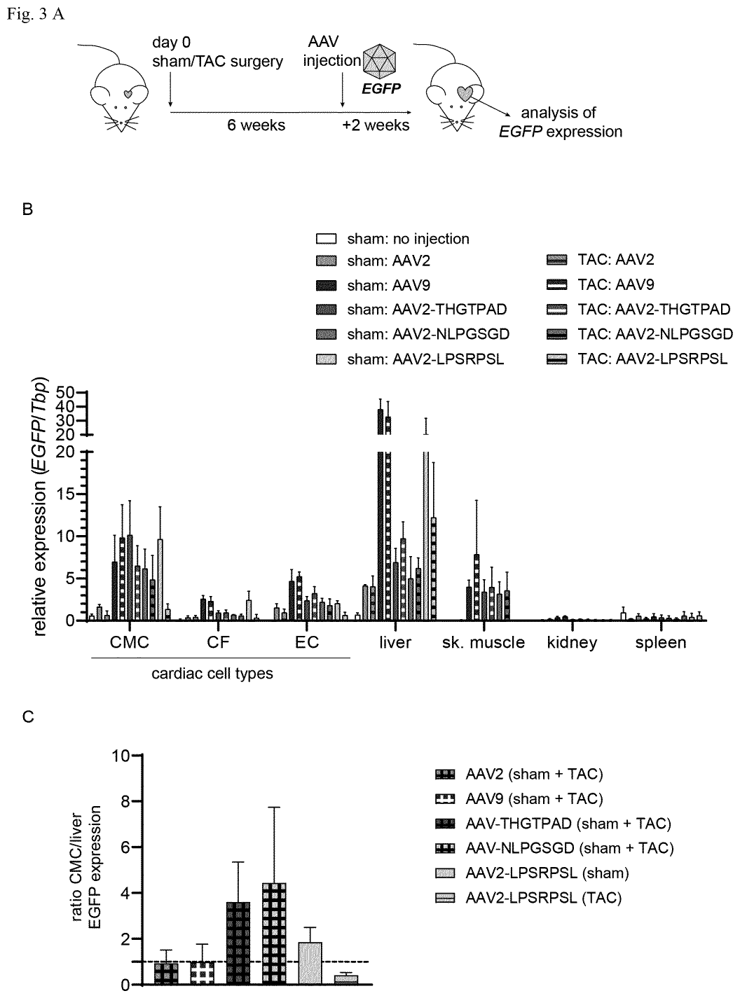

FIG. 3 shows transduction efficiency and specificity of viral vector particles according to the invention, and

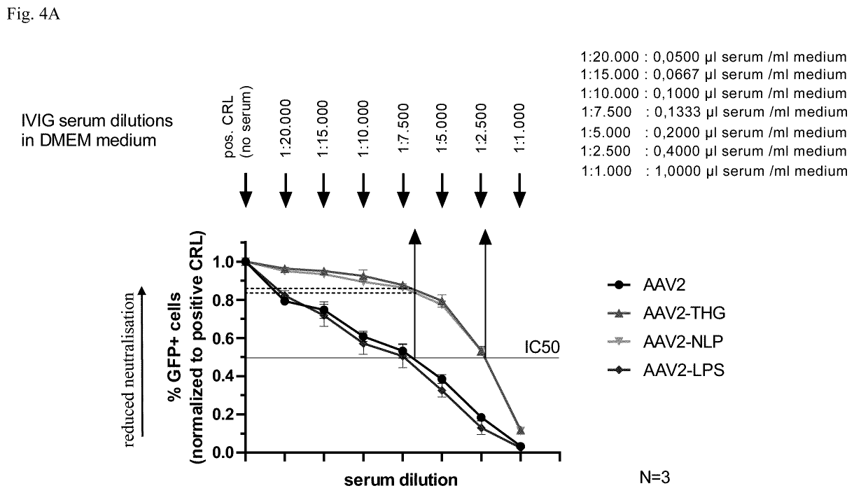

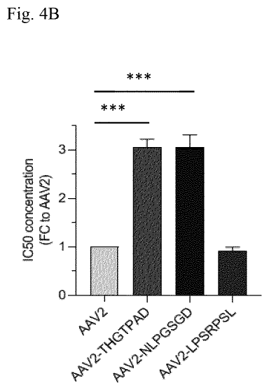

FIG. 4A and B show results for measuring neutralisation of viral vector particles according to the invention by human intravenous immunoglobulin (IVIG).

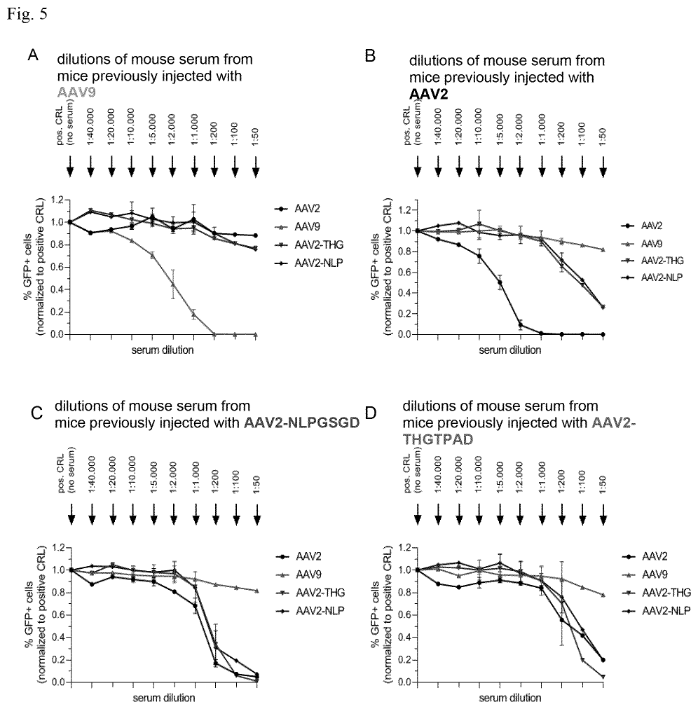

FIG. 5A to D show results for measuring neutralisation of viral vector particles according to the invention by serum from mice which were previously injected with either AAV2, AAV9 or viral vector particles according to the invention.

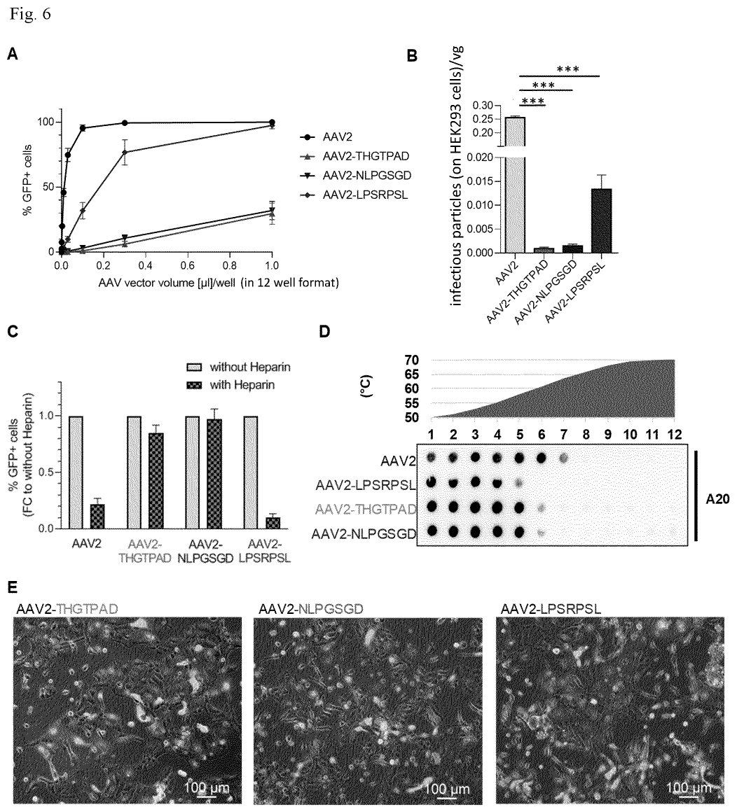

FIG. 6A and 6B show results of transduction efficiency by viral vectors in non-cardiomyocytes,

FIG. 6C shows results of transduction efficiency by viral vectors in presence or absence of heparin,

FIG. 6D shows thermal stability data for viral vectors,

FIG. 6E show cardiomyocyte transduction by viral vectors, and

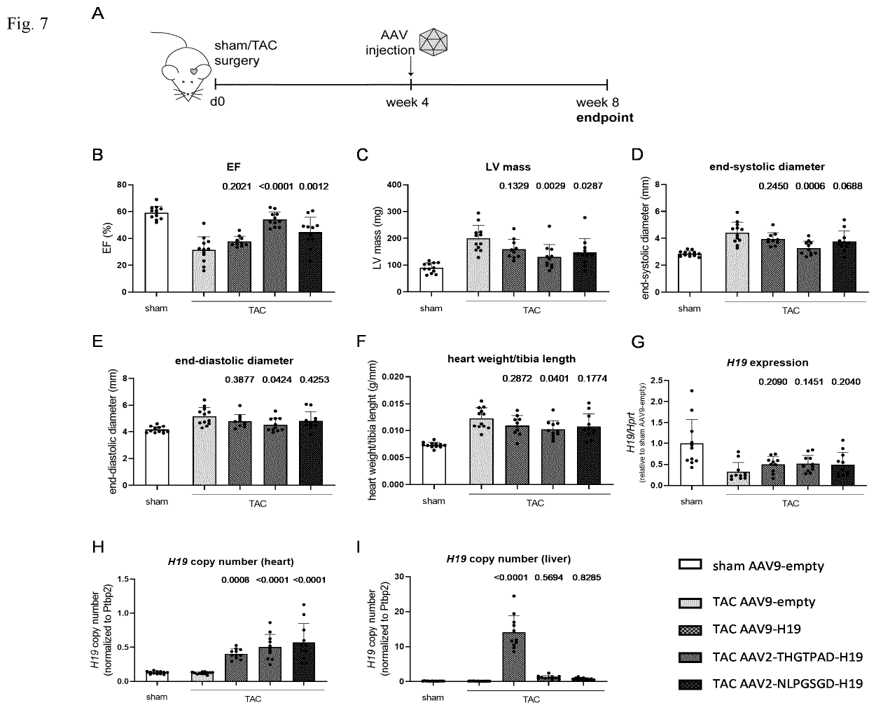

FIG. 7A shows a scheme of the experimental treatment,

FIG. 7B to E show echocardiographic results and Fig. F shows heart to tibia length rations, Fig. G shows expression levels of H19 and (H-I) vector copy number of H19 in heart and liver tissue of sham- and TAC-operated mice of in vivo effects of viral vectors of the invention and of comparative vectors.

### Example: Identification of AAV2 Viral Vector Particles Having Specificity for Cardiomyocytes

Generally, an initial library of viral particles of AAV2 capsid-mutants was generated, which contained random amino acid sections inserted into CAP. The inserted amino acid sections were encoded by nucleic acid constructs arranged in a wild-type gene encoding CAP between the sections encoding amino acids No. 587 and No. 588 of the wild-type sequence. Between the codons for amino acid 587 and for amino acid 588 of the wild-type CAP the nucleic acid constructs from 5′ to 3′ encoded inserted amino acid sequences consisting of the coding sequence for AAA as a linker sequence, a random 7-mer as the inserted amino acid sequence, and the coding sequence for AA as a linker sequence, resulting in the arrangement of the respective encoded amino acid sequences from N-terminus to C-terminus.

For selection of cardiomyocyte specific viral vector particles, 2 to 4 mice (male C57BL/6N, 6 to 8 weeks old) were used in which cardiac pressure overload was artificially induced as described by Rockman et al., PNAS 88: 8277-8281 (1991), which mice are also referred to as TAC-operated mice, using a 26-gauge needle. Aortic stenosis by the resultant transverse aortic constriction (TAC) was confirmed by echocardiography prior to injection of viral/vector particles. Mice were once treated by tail vein injection with 0.66×10¹¹ to 1×10¹¹ viral/vector particles.

It is assumed that the use of TAC-operated mice in the in vivo selection process as this mouse model supported the identification of capsid-mutant viral vector particles of AAV2 which in the CAP contained an inserted amino acid section which results in an increased tropism especially for hypertrophic cardiomyocytes. Accordingly, the viral vector particles are suitable for use in treatment of cardiac tissue, especially for targeting cardiomyocytes in the hypertrophy disease stage. At the same time, the in vivo selection in mice allows to identify capsid-mutant AAV particles which have a reduced tropism for liver and thus results in more efficient cardiomyocyte-specific transgene delivery and transgene expression, e.g. increased in comparison to wild-type AAV9. In this process, for the initial library, viral particles containing Rep and Cap genes were used, in the further selection process, Rep coding sequence was replaced by expression cassette for EGFP DNA.

As a representative transgene, the coding sequence for EGFP (enhanced green fluorescent protein) was used.

The AAV library of viral vector particles was produced from a plasmid pool by calcium phosphate transfection of HEK293 cells followed by iodixanol gradient purification of viral vector particles. Vector particle titers were determined by quantitative PCR using cap-specific or EGFP-specific primers.

The in vivo selection process consisted of three consecutive selection steps. In the first selection step, the initial library of viral particles was injected into mice 53 d after TAC surgery. 3 d later, cardiac tissue was fractionated to isolate cardiomyocytes. For this, mice were anaesthetized in an inhalation chamber with 4% isoflurane in oxygen. The animals were fixed in the supine position on a hot plate (at 37° C.) and anesthesia was maintained with a respiratory mask (2% isoflurane in oxygen). The skin was incised and an incision was made between two tracheal trabeculae, through which a cannula was inserted. The tube was then fixed with a thread and connected to an artificial ventilation system. After disinfection of the thorax, the skin was cut along a length of 2-3 cm parallel to the rib arch, the abdomen and thorax were opened and any bleeding was dabbed. Next, the aorta was localized, lifted and gently cut. A blunt cannula was inserted through the hole and fixed with a thread. The heart was immediately retrograde perfused with pre-warmed perfusion buffer (113 mM NaCl, 4.7 mM KCl, 0.6 mM KH₂PO₄, 0.6 mM Na₂HPO₄, 1.2 mM MgSO₄—7H₂O, 0.032 mM Phenol Red, 12 mM NaHCO₃, 10 mM KHCO₃, 10 mM HEPES, 30 mM Taurine, 0.1% Glucose, 10 mM 2,3-Butanedione monoxime) for 3 min within the mouse, then removed from the mouse and perfused for additional 3 min with perfusion buffer followed by 10 min perfusion with pre-warmed digestion buffer (113 mM NaCl, 4.7 mM KC1, 0.6 mM KH₂PO₄, 0.6 mM Na₂HPO₄, 1.2 mM MgSO₄—7H₂O, 0.032 mM Phenol Red, 12 mM NaHCO₃, 10 mM KHCO₃, 10 mM HEPES, 30 mM Taurine, 0.1% Glucose, 10 mM 2,3-Butanedione monoxime, 12.5 μM CaCl₂, 700 U/ml Collagenase II). The atriums were removed and the ventricles were dissociated mechanically by cutting in 2.5 ml warm digestion buffer and shearing through a 1 ml syringe. Collagenase II digestion was stopped by adding 2.5 ml stop buffer (113 mM NaC1, 4.7 mM KCl, 0.6 mM KH₂PO₄, 0.6 mM Na₂HPO₄, 1.2 mM MgSO₄—7H₂O, 0.032 mM Phenol Red, 12 mM NaHCO₃, 10 mM KHCO₃, 10 mM HEPES, 30 mM Taurine, 0.1% Glucose, 10 mM 2,3-Butanedione monoxime, 12.5 μM CaCl₂, 10% FBS) to the cell suspension. The obtained cell suspension was filtered through a 100 μm cell strainer and the filter was washed with 1-2 ml with AMCF medium (10.8 g/l MEM HBS with NEAA (Bioconcept), 4.2 mM NaHCO₃, 2ng/ml vitamin B12, 1% penicillin/streptomycin (100 U/ml; 100 μg/ml), 10% FBS, pH 7.3). The appearance of rod-shaped cardiomyocytes was assessed under the microscope. Cardiomyocytes were sedimented for 10 min at room temperature (RT). The cardiomyocyte sedimentation pellet (→CMC fraction) was washed in phosphate buffered saline (PBS), centrifuged for 5 min at 900×g at 4° C., frozen in liquid nitrogen and stored at −80° C. The remaining supernatant was centrifuged for 3 min at 30×g at RT to remove residual cardiomyocytes. The cell pellet containing the residual cardiomyocytes was discarded and the supernatant containing the remaining other cardiac cell types was further processed. During the library selection, when no other specific cardiac cell types, e.g. fibroblasts or endothelial cells were required, the supernatant was centrifuged at 430×g to pellet all non-cardiomyocytes and the pellet was frozen in liquid nitrogen and stored at −80° C. If also cardiac fibroblast and endothelial cells were required (for vector copy and expression analysis of the individual vector variants), the cell pellet containing the non-myocyte fraction was instead dissolved in AMCF medium (10.8 g/l MEM HBS with NEAA (Bioconcept), 4.2 mM NaHCO₃, 2 ng/ml vitamin B12, 1% penicillin/streptomycin (100 U/ml; 100 μng/ml), 10% FBS, pH 7.3) and pre-plated on a 10 cm petri dish in a 1% CO₂ incubator for 1 h. The attached cells (→cardiac fibroblasts) were washed with PBS twice, then 2 ml PBS were added to the dish and cells were harvested with a cell scraper, centrifuged at 900×g for 5 min at 4° C. The pellet was frozen in liquid nitrogen and stored at −80° C. The supernatant of the pre-plating step, containing the non-myocyte, non-fibroblast fraction, was next centrifuged at 430×g for 5 min at 4° C. The resulting cell pellet was resuspended in 80 μl MACS buffer (MACS bovine serum albumin stock solution diluted 1:20 in auto-MACS rinsing solution, both from Miltenyi Biotec) mixed with 20 μl CD146 MACS beads (Miltenyi Biotec) and incubated for 15 min at 4° C. Afterwards, 2 ml of MACS buffer was added, the cell suspension was mixed thoroughly and centrifuged for 5 min at 430×g at 4° C. The cell pellet was resuspended in MACS buffer and transferred to a pre-washed MACS separating column. After three washing steps with MACS buffer, the separation columns were removed from the magnetic field. The EC fraction was collected by rinsing the column three times with 500 μl MACS buffer and centrifuged at 900×g for 5 min at 4° C. Pellet was frozen in liquid nitrogen and stored at −80° C.

DNA was isolated from the cell fractions using the DNeasy Blood and Tissue kit (obtained from Qiagen, Hilden, Germany) according to the manufacturer's instructions. Viral vector DNA was amplified by PCR using primers that flank the coding sequence of the inserted amino acid sequence (forward primer SEQ ID NO: 57, reverse primer SEQ ID NO: 58) for re-cloning the inserted amino acid sequences of the CAP gene of the viral particles that were accumulated in cardiomyocytes, and a secondary library of viral vector particles was generated from these re-cloned CAP gene sequences.

For the second selection step, in the secondary library, the rep gene was replaced by an expression cassette encoding EGFP, and during the production of the viral vector particles of the secondary library, the rep protein-coding sequence was supplied in trans by plasmid transfection. The rep encoding nucleotide sequence was provided on a separate plasmid which was additionally used during transfection for AAV vector production. For packaging, the vector genome of the secondary library was flanked by inverted terminal repeats (ITRs) of AAV2. The viral vector particles of the secondary library were injected 42 d after TAC surgery, and cardiomyocytes and non-myocyte cells were collected two weeks later, followed by subsequent amplification of DNA for re-cloning the nucleic acid sequences encoding the inserted amino acid sequences which were further accumulated in cardiomyocytes. The re-cloned amino acid sequences were used to generate a tertiary library which was selected the same way as the previous second selection round.

The selection process is depicted in FIG. 1, wherein the target cells are cardiomyocytes, and liver tissue was analysed as the main off-target tissue of AAV vectors for evaluating specificity for cardiomyocytes.

After the three rounds of selection, the sub-library was isolated from the cardiomyocyte fraction, from the non-myocyte cardiac fraction, and from the liver tissue. The DNA of this sub-library after three selection rounds was analysed by next-generation sequencing on the 454-pyrosequencing platform (GS Junior, Roche Diagnostics), using a cap-specific primer (forward primer of SEQ ID NO: 57). Sequencing data identified coding sequences of cardiomyocyte-enriched variants that in the cap gene encoded inserted amino acid sequences of SEQ ID NO: 1 to SEQ ID NO: 53.

For production of individual capsid-modified vector particles which were selected from the previously conducted selection of the AAV peptide display library, oligonucleotides encoding the inserted amino acid sequences and flanking portions were used to individually generate viral vector particles having a cap protein containing one of the inserted amino acid sequences. The oligonucleotides for individual cap genes encoding one of the inserted amino acid sequences were cloned into the helper plasmid pRC'99 as described by Zhang et al., Hum. Gene Ther., 1284-1296 (2019).

The viral vector particles according to the invention which contained CAP with an inserted amino acid section of SEQ ID NO: 1 had a CAP of SEQ ID NO: 54, and the CAP with an inserted amino acid section of SEQ ID NO: 11 had a CAP of SEQ ID NO: 55. AAV viral vector particles, both wild-type and the capsid-modified variants including a CAP with an inserted amino acid section according to the invention, and also wild-type AAV9 particles, were produced by calcium phosphate transfection of HEK293 cells, with subsequent purification of viral vector particles by iodixanol gradient purification. The individual viral vectors each containing one of the inserted amino acid sequences were produced as viral vectors encoding for EGFP under the control of the CMV promoter in a self-complementary genome conformation (scEGFP). In short, the process comprised the steps of transfecting the vector genome plasmid (CMV promoter and EGFP coding sequence, flanked by AAV2 ITRs), the respective helper plasmid (containing the cap gene, if applicable with an inserted amino acid section, and the rep gene of wild-type AAV2) and an adenoviral helper plasmid (containing adenoviral helper functions required for AAV vector production) in HEK293 cells for AAV vector production, with subsequent purification of vector particles by iodixanol gradient centrifugation.

Genomic titers of vector productions were determined by quantitative PCR using primers specific for the EGFP encoding sequence.

The copy numbers of viral vector particles present in different tissues of experimental animals on the example of CAP containing inserted amino acid section of SEQ ID NO: 1 or SEQ ID NO: 11 show that the viral vector particles containing a CAP according to the invention have higher specificity for cardiac myocytes, e.g. compared to wild-type AAV9, specifically in relation to liver cells.

AAV vector copy number analysis was performed by determining the absolute gene copy number of Ptbp2 (polypyrimidine tract binding protein 2; two copies per diploid genome) and EGFP via qPCR in cardiac cell samples and organ tissue, respectively, using the absolute standard curve method. Multiplex TaqMan probe based qPCR detection was performed in a 384-well format using the TaqMan Fast Advance Master Mix (Thermo Fisher Scientific), the TaqMan Copy Number Assay for EGFP (Thermo Fisher Scientific; FAM-fluorescent labeled), Ptbp2 primer (forward: TCTCCATTCCCTATGTTCATGC (SEQ ID NO: 59), reverse: GTTCCCGCAGAATGGTGAGGTG (SEQ ID NO: 60)) and a JOE-fluorescent labeled Ptbp2 probe (5′ [JOE]-ATGTTCCTCGGACCAACTTG-[BHQ1] 3′ (SEQ ID NO: 61)). Each qPCR reaction contained a final concentration of 1× TaqMan Fast Advance Master Mix, 1× EGFP TaqMan Copy Number Assay, 150 nM Ptbp2 TaqMan probe, 330 nM primer (forward and reverse) and 2 μl of DNA sample in a total volume of 10 μl. DNA samples comprehended either linearized plasmid DNA (containing one copy of Ptbp2 and EGFP per plasmid) for an absolute copy number standard curve (5×10⁵, 5×10^{4,} 5×10^{3,} 5×10², 5×10¹ molecules/μl) or pre-diluted DNA with concentrations of 15 ng/μl.

The qPCR was run on a QuantStudio Real-Time PCR System (ThermoFisher Scientific) using the following protocol: initial activation at 50° C. for 2 min and 95° C. for 20 sec, followed by 40 cycles of denaturation at 95° C. for 5 sec, primer/probe annealing and elongation at 56° C. for 20 sec and detection of the fluorescence signal at 65° C. for 20 sec. The vector copy number (VCN) in diploid cells was calculated by the following formula:

VCN=quantity(EGFP)/quantity(Ptbp2)×2.

The expression levels of EGFP, representing an effector gene, show that the viral vector particles according to the invention have high specificity of expression of the effector gene in cardiomyocytes. The level of transgene expression is comparable to the expression level of the same transgene delivered by wild-type AAV9. However, in comparison to AAV9, the vector particles according to the invention show reduced expression in liver tissue, which is the main off-target.

The expression of the exemplary transgene EGFP was analysed by quantitative reverse transcription PCR (qrtPCR) in cardiac cell types that were obtained by heart fractionation and in liver tissue which were collected 2 weeks after injection of the viral vectors. The results were generally normalized to the RNA input in relation to the transcripts of the TATA-box binding protein (Tbp). In detail, Reverse transcription of 65-500 ng total RNA was performed using the Biozym cDNA Synthesis Kit (Biozym) according to the manufacturer's instruction. In case of organ tissue samples, a second DNase digestion was performed directly prior reverse transcription by incubating 500 ng total RNA with 0.684 μl DNase (1:10 dilution, RNase-Free DNase Set (Qiagen)), 1.15 μl RDD buffer (RNase-Free DNase Set (Qiagen)) and 0.144 μl RNasin Ribocluclease Inhibitor (Promega) in a total volume of 11.5 μl for 30 min at 37° C. The reaction was stopped by adding 0.23 μl 62.5 mM EDTA and incubation for 5 min at 65° C. RNA dilutions of 11.5-11.73 μl containing 65-500 ng RNA (with or without second DNase digest) were reverse transcribed using 4 μl 5×cDNA synthesis buffer, 2 μl dNTP Mix (10 mM each), 1 μl hexamer primer (25 μM), 0.5 μl RNase inhibitor (40 U/μl) and 1 μl reverse transcriptase. The reaction mix was incubated at 30° C. for 10 minutes, followed by 60 min at 55° C., and finally, the enzyme was heat inactivated at 99° C. for 5 min. Prior to qPCR, cDNA samples were diluted with two volumes of nuclease-free H₂O (1:3 dilution) and stored at −20° C.

qPCR measurements were performed in a 384-well format using the iQ SYBR Green Supermix (Biorad) according to the manufacturer's instructions. The reaction mix composed of 5 μl iQ SYBR Green Supermix, 0.05 μl of a ROX Reference Dye 1:50 dilution (Thermo Scientific), 0.025 μl Precision Blue™ Real-Time PCR Dye (BioRad), 0.5 μl of pre-mixed primer (10 μM forward and 10 μM reverse primer), 2.45 μl nuclease-free H₂O and 2 μl of cDNA (1:3 dilution after cDNA synthesis) was mixed and the qPCR protocol was run on a Viia™ 7 Real-Time PCR System (ThermoFisher Scientific) using the following protocol: initial activation at 95° C. for 3 min, 45 cycles of denaturation at 95° C. for 15 seconds, primer annealing at 60° C. for 30 seconds, elongation at 72° C. for 40 seconds, followed by the generation of a melting curve with fluorescence detection very 0.5° C. from 95° C. to 5 ° C. for 10 seconds to ensure amplification of a single PCR amplicon. EGFP expression in cardiac cell fractions and murine organs was analyzed by the relative standard method. The same 1:5 dilution series of pooled EGFP expression samples was included in all qPCR measurements in order to obtain relative standard values for all samples.

FIG. 2 shows average copy numbers (VCN) of viral vector particles in cells and organs, respectively, isolated from experimental animals, namely in cardiomyocytes (CMC), liver, skeletal muscle (sk. muscle), in kidney and spleen. The viral vector particles indicated from top to bottom are shown in the graph from left to right. The animals are indicated as sham-operated (sham) or with surgically induced transverse aortic constriction (TAC). The results show that the CAP containing the inserted amino acid section THGTPAD (SEQ ID NO: 1, AAV2-THGTPAD) and the CAP containing the inserted amino acid section NLPGSGD (SEQ ID NO: 11, AAV2-NLPGSGD) give similar copy numbers in cardiomyocytes as wild-type AAV9 (AAV9) and higher copy numbers than wild-type AAV2 (AAV2), and in liver give lower copy numbers than wild-type AAV2 and AAV9. This shows an increased specificity of CAP containing an inserted amino acid section according to the invention for cardiomyocytes over the wild-type AAV serotypes AAV2 and AAV9. In contrast, CAP with an inserted amino acid section having sequence LPSRPSL (SEQ ID NO: 62, comparative) has a lower copy number in cardiomyocytes and a higher copy number in liver, indicating a lower tropism for cardiomyocytes.

The transduction efficiency and transduction specificity of viral vector particles according to the invention were analysed by introducing viral vectors having a CAP with an inserted amino acid section of SEQ ID NO: 1 (CAP of SEQ ID NO: 54) or SEQ ID NO: 11(CAP of SEQ ID NO: 55), and for comparison wild-type AAV2 or wild-type AAV9 or a CAP containing inserted amino acid section of SEQ ID NO: 62. The viral vector particles contained the expression cassette for EGFP. The viral vector particles were injected into sham-operated mice or TAC-operated mice (each 2 to 4 animals) 6 weeks after surgery. After two weeks following the viral vector particle injection, expression of EGFP was determined by qrtPCR in cardiomyocytes, cardiac fibroblasts (CF) and cardiac endothelial cells (EC) that were fractionated from cardiac tissue, in skeletal muscle cells (sk. muscle), in liver, kidney and spleen.

FIG. 3A schematically depicts the analytical process. FIG. 3 B shows the results of detecting expression of EGFP, normalized to expression of Tbp (relative expression (EGFP/Tbp), for the sham-operated mice (sham) or TAC-operated mice (TAC) separately for each vector: wild-type AAV2 (AAV2), wild-type AAV9 (AAV9), viral vector particle with CAP containing inserted amino acid section of SEQ ID NO: 1 (AAV2-THGTPAD), viral vector particle with CAP containing inserted amino acid section of SEQ ID NO: 11 (AAV2-NLPGSGD), or comparative vector particle with CAP containing inserted amino acid section of SEQ ID NO: 62 (AAV2-LPSRPSL). These experimental combinations are indicated in FIG. 3B from top to bottom in two columns, and are indicated in this order from left to right for the different cell-types. The results show that the vector particles according to the invention in cardiac myocytes generate a higher expression of the transgene than wild-type AAV2 and a similar expression than wild-type AAV9 in cardiac myocytes of sham- and TAC-operated mice, and a lower expression of the transgene in liver, the main off-target organ, than wild-type AAV9.

FIG. 3C shows the expression of EGFP, normalized to expression of Tbp, in cardiac myocytes (CMC) relative to expression in liver, combined for the results from sham-operated animals and TAC-operated animals for the vectors AAV2, AAV9, AAV2-THGTPAC and AAV2-NLPGSGD, and separate for sham and TAC for the CAP containing comparative inserted amino acid section SEQ ID NO: 62 (AAV2-LPSRPSL). The data are given for AAV2 and AAV9 (wild type capsids), and for viral vector particles with CAP containing inserted amino acid section of SEQ ID NO: 1 (AAV2-THGTPAD), and vector with CAP containing inserted amino acid section of SEQ ID NO: 11 (AAV2-NLPGSGD), as well as for comparative vector (SEQ ID NO: 62). Based on the exemplary CAP containing inserted amino acid sections this shows that the viral vector particles on the basis of AAV2 containing CAP with an inserted amino acid section according to the invention results in significantly increased expression specificity of the effector molecule encoded by the viral vector particle in cardiac myocytes, e.g. significantly increased in comparison to wild-types AAV2 and AAV9, and in comparison to comparative CAP including inserted amino acid section of SEQ ID NO: 62.

For testing the persistence of viral vector particles according to the invention in blood serum containing human intravenous immunoglobulin (IVIG), the neutralisation activity of IVIG serum was tested. Viral vector particles containing CAP according to the invention as well as vector particles of the wild-type serotype AAV2 were incubated with different dilutions of the IVIG serum in 1 mL DMEM cell culture medium (supplemented with 10% fetal bovine serum and 1% penicillin/streptomycin). The viral vector particles were used at a concentration which in the absence of IVIG, i.e. in the cell culture medium only, transfects 30 to 40% of cells to express EGFP (30 to 40% of cells EGFP positive), which was also used as a positive control. The viral vector particles were incubated in the cell culture medium in mixture with or without IVIG serum for 1 h at room temperature and then added to HEK293 cells cultivated in 12-well plates. After an incubation of 48 hours under cell culture conditions, the HEK293 cells were analysed by fluorescence activated cell sorting (FACS) to determine the percentage of EGFP-positive cells.

FIG. 4A shows the FACS results, wherein the percentage of EGFP-positive cells was normalized to the percentage determined for the positive control (pos. CRL)=1. It is found that the wild-type AAV2 (AAV2) and the comparative viral vector particle containing CAP with SEQ ID NO: 62 as the inserted amino acid section (AAV2-LPS) are neutralized already at higher dilutions of the IVIG serum than viral vector particles of the invention, e.g. containing the inserted amino acid section of SEQ ID NO: 1 (AAV2-THG) or containing the inserted amino acid section of SEQ ID NO: 11 (AAV2-NLP). FIG. 4B shows a graph of the IC50 values derived from the data of FIG. 4A.

The production of viral vector particles according to the invention was performed as described herein for the expression and vector copy number analysis in mouse tissue.

FIG. 5A to D show the FACS results, wherein the percentage of EGFP-positive cells was normalized to the percentage determined for the positive control (pos. CRL)=1. It is found that mouse serum from mice which were previously injected with wild-type AAV9 (AAV9) only neutralise AAV9, whereas AAV2 and the viral vector particles of the invention transduce cells despite the presence of this serum. Similar, mouse serum from mice which were previously injected with wild-type AAV2 (AAV2) only neutralise AAV2, whereas AAV9 and the viral vector particles of the invention remain their ability to transduce cell. Of note, serum of mice previously injected with AAV-NLP shows poor neutralisation capacity, resulting in neutralisation of AAV2 and the viral vector particles of the invention only at low serum dilutions. Similar, serum of mice previously injected with AAV-THG shows poor neutralisation capacity, resulting in weak neutralisation of only wild-type AAV2 at low serum dilutions.

An in vitro characterization of AAV2 viral particles of the invention is shown in FIG. 6. Different volumes of vectors were used for transduction of Hek293 cells in a 12-well format. Genomic titers of vectors used in this assay were in a comparable range. Transduction efficiency was determined by fluorescence activated cell sorting (FACS) for EGFP positive cells 48 h after transduction (n=3). FIG. 6A show the transduction efficiency of AAV2 viral vectors of the invention AAV2-THGTPAD (containing the inserted amino acid section of SEQ ID NO: 1) and AAV2-NLPGSGD (containing the inserted amino acid section of SEQ ID NO: 11) and, as a comparison, wild-type AAV2 and AAV2-LPSRPSL (containing the comparative inserted amino acid section of SEQ ID NO: 62). On the basis of the data of FIG. 6A, FIG. 6B shows the calculated ratio of infectious particles (on Hek293 cells) per vector genome, wherein genomic titer was determined by qPCR (quantitative PCR). The results show that in these cells, the comparative vector AAV2-LPSRPSL shows a lower infectivity than wild-type AAV2, and that vectors according to the invention show a severely reduced infectivity in these non-target cells.

FIG. 6C shows the result of a heparin competition assay, wherein heparin forms a soluble analogue of HSPG, which is a putative receptor for viral vectors. AAV vectors and the wild-type AAV2 were pre-incubated with heparin prior transduction of Hek293 cells in order to investigate heparin sulfate proteoglycan (HSPG) dependent cell entry. Transduction efficiency was determined by FACS for EGFP positive cells 48 h after transduction (n=3). The result shows that heparin did not affect transduction by the vectors according to the invention, but did affect transduction by comparative vectors AAV2 and AAV2-LPSRPSL. This suggests that cell entry of vectors of the invention occurs independent from HSPG.

The thermal stability of AAV vector particles was assayed by subjecting the vector particles to different temperatures for 15 min followed by dot blotting using an A20 antibody for detection, which is specific for detection of assembled capsid proteins of intact AAV vector particles. The result is shown in FIG. 6D, showing that the stability of the capsid is lower for both the comparative vector AAV2-LPSRPSL and viral vectors of the invention than for the wild-type AAV2. However, comparative vector AAV2-LPSRPSL was the least stable variant, showing partial degradation at 57.9° C., while vectors of the invention showed a very similar stability with a partial capsid degradation starting at 60.7° C., and initiation of degradation of the variant capsid initiated only at 63.4° C.

FIG. 6E shows the result of assaying cross-species activity. Human induced pluripotent stem-cell derived cardiomyocytes (iPSC-CMCs) were transduced with AAV vectors expressing scEGFP with a vector particle-to-cell-ratio of 2×10³. EGFP expression was assessed by fluorescence microscopy (scale bar=100 μm) at 7 days after transduction (n=3). The results show that the viral vectors of the invention transduce cardiomyocytes.

The following mouse experimental data show the therapeutic efficacy and liver de-targeting of viral vectors of the invention on the examples of AAV2-THGTPAD and AAV2-NLPGSGD. Utilizing the observation that AAV9-based delivery of long non-coding RNA (lncRNA) H19 reverses pathological cardiac hypertrophy in the TAC mouse mode, H19 was packaged into AAV9, AAV2-THGTPAD and AAV2-NLPGSGD and injected mice 4 weeks after induction of TAC, as schematically depicted in FIG. 7A. The data show that due to the improved cardiomyocyte tropism low viral vector doses suffice for transduction, as only 3.55×10¹⁰ viral genomes (vg)/mouse were injected. Functional assessment by echocardiography 4 weeks after AAV treatment showed significant rescue in left ventricular ejection fraction for both AAV2-THGTPAD-H19 and AAV2-NLPGSGD-H19 but not for AAV9-H19 in comparison to the AAV9-empty control group (FIG. 7 B). This was concomitant with lower left ventricular mass (FIG. 7C) and rescued cardiac dimensions (FIG. 7D, 7E), as well as lower heart weight to tibia length ratio (FIG. 7 F) in AAV2-THGTPAD-H19 and AAV2-NLPGSGD-H19 treated mice. The AAV9-H19 treatment group showed a clear trend toward therapeutic rescue for all parameters but this did not reach statistical significance. H19 expression analysis after heart explanation showed the expected reduction of H19 in cardiac hypertrophy which was only partially rescued in AAV2-THGTPAD-H19, AAV2-NLPGSGD-H19 and AAV9-H19 treated mice (FIG. 7G, presumably owing to the low vector dose in conjunction with dilution effects of non-cardiomyocytes). Nevertheless, H19 copy number analysis showed a strong increase for all variants compared to AAV9-empty controls (FIG. 7H). Strikingly, while AAV9-H19 strongly accumulated in the liver, H19 copy numbers in AAV2-THGTPAD-H19, AAV2-NLPGSGD-H19 treated mice were comparable to the sham and AAV9-empty control groups (FIG. 7I).

1. AAV2 viral vector particle comprising a nucleic acid construct for an effector molecule, comprising a capsid protein (CAP) which C-terminally to amino acid No. 587 or No. 588 or No. 453 of the wild-type amino acid sequence of CAP of SEQ ID NO: 56 contains an inserted amino acid section comprising one of the amino acid sequences selected from SEQ ID NO: 1 to SEQ ID NO: 53.

2. AAV2 viral vector particle according to claim 1, wherein a linker sequence of 1 to 4 amino acids is arranged between amino acid No. 587 or No. 588 or No. 453 of the N-terminal section of CAP and the N-terminal amino acid of the inserted amino acid section.

3. AAV2 viral vector particle according to claim 1, wherein a linker sequence of 1 to 3 amino acids is arranged between the C-terminus of the inserted amino acid section and the N-terminal amino acid of the remaining C-terminal portion of CAP.

4. AAV2 viral vector particle according to claim 1, wherein remaining C-terminal portion of CAP are amino acids 588 to 735, or amino acids 589 to 735, or amino acids 454 to 735, of SEQ ID NO: 56.

5. AAV2 viral vector particle according to claim 1, comprising mutations R585A (amino acid 585 Arg to Ala) and R588A (amino acid 588 Arg to Ala).

6. AAV2 viral vector particle according to claim 1 for use in the treatment of a disease or defect of cardiac myocytes, or in the treatment of a disease or defect of muscular myocytes or of skeletal muscle cells.

7. AAV2 viral vector particle for use in the treatment of a disease or defect of cardiomyocytes according to claim 6, wherein the vector particle contains a nucleic acid construct comprising an effector sequence.

8. AAV2 viral vector particle for use in the treatment of a disease or defect of cardiomyocytes according to claim claim 7, wherein the effector sequence is an expression cassette encoding an effector molecule.

9. AAV2 viral vector particle for use in the treatment of a disease or defect of cardiomyocytes according to claim 6, wherein the disease or defect is cardiac hypertrophy, myocardial infarction, cardiotoxicity or cardiac failure.

10. AAV2 vector particle for use in the treatment of a disease or defect of cardiomyocytes according to claim 6, wherein the treatment is in vivo or ex vivo transduction of cardiomyocytes.

11. AAV2 viral vector particle for use in the treatment of a disease or defect of cardiomyocytes according to claim 6, wherein the person receiving the viral vector particle has antibody neutralizing wild-type AAV2 and/or has antibody neutralizing AAV9.

12. Process for producing AAV2 viral vector particles by delivery of components for AAV vector production in a cultivated eukaryotic cell, followed by cell lysis and removal of cellular components and plasmid DNA, and further purification of AAV viral vector particles, wherein the AAV2 viral vector particle comprises a capsid protein (CAP) which C-terminally to amino acid No. 587 or C-terminally to amino acid No. 588 or C-terminally to amino acid No. 453 of the wild-type amino acid sequence of CAP contains an inserted amino acid section comprising one of the amino acid sequences selected from SEQ ID NO: 1 to SEQ ID NO:

53.

13. Method of treatment of cardiac diseases, including cardiac defects, comprising administering an AAV2 vector particle according to claim 1 to a patient who is diagnosed to have a cardiac disease or cardiac defect.
